(19) United States
(12) Patent Application Publication
(10) Pub. No.: US 2023/0130749 A1
(43) Pub. Date: Apr. 27, 2023

(54) THREAD INSERTION DEVICE

(71) Applicant: ALLERGAN PHARMACEUTICALS INTERNATIONAL LIMITED, Dublin (IE)

(72) Inventors: David Dadi SEGAL, Tel Aviv (IL); Liat GOLDSHAID-ZMIRI, Petach Tikva (IL); Lital SHKLANOVSKY, Kiryat Ono (IL); Eran GOLDBERG, Ramat Gan (IL); Yehonatan COHEN, Karnei Shomron (IL); Naor MARCIANO, Ashdod (IL)

(21) Appl. No.: 17/908,121

(22) PCT Filed: Mar. 1, 2021
(86) PCT No.: PCT/EP2021/055027

§ 371 Date: Aug. 30, 2022

(30) Foreign Application Priority Data
Mar. 1, 2020 (IL) 273004

(51) Int. Cl. A61B 17/34; A61M 5/32

(52) U.S. Cl. CPC A61B 17/3468; A61M 5/322; A61B 2017/00792

(57) ABSTRACT

The disclosure relates to a device for creating an intradermal channel in a skin tissue of a patient, with a thread. The device can include a main body, a penetration element, and a displacement mechanism. The main body can have a lumen. The penetration element can be at a distal end of the main body and have a cannula which is configured to contain the thread in its entirety. The displacement mechanism can have at least a portion thereof permanently contained within the lumen of the main body. The displacement mechanism can be configured to effect a relative motion between the cannula and the thread, thereby to expose at least a portion of the thread to a patient's tissue within an intradermal channel.

### CROSS-REFERENCE TO RELATED APPLICATIONS

This application claims priority to and benefit from Israel Patent Application No. 273004, filed on Mar. 1, 2020, the entirety of which is incorporated herein by reference.

### FIELD

The disclosure relates in general to the field of medical devices. More specifically, the disclosure relates to the field of medical devices that are used for the insertion and placement of a thread at an intradermal location within a human organ.

### BACKGROUND

In the field of cosmetic procedures and plastic surgeries, a necessity frequently arises for the intradermal insertion and stretch of a thread at a patient's organ. Such a necessity arises, for example, in a face-lifting surgery or procedure, where a thread is inserted under or into the skin of the patient. Hereinafter, and unless otherwise specifically indicated, the device of the disclosure may be used in any intradermal procedure which requires the insertion of a thread into an intradermal location.

Several devices have been developed for an intradermal insertion of a thread. For example, CN 205,215,280, KR 10-2014-0029,007 SG 188,050, and KR 10-170-3305 describe devices for a thread-insertion, particularly for use in a cosmetic procedure commonly known as PDO which is used for face-lifting. WO 2017/031478, and US 2014/155,913 describe other thread-insertion devices. All these thread-insertion devices typically include a penetration element (such as a needle and/or cannula) for use with a thread which is at least partially maintained outside of the penetration element prior to the insertion. In several of these devices an additional portion of the thread (other than the thread-portion which is maintained outside of the penetration element) is kept in a hollow channel within the penetration element. The insertion procedure by the prior-art devices is performed by: (a) inserting the penetration element sub-dermally, together with the thread attached to it (in a manner as described), thereby to form an intradermal channel; and (b) upon completion of the channel, withdrawing the penetration element, while the thread is stretched and remains within the intradermal channel due to friction between the externally exposed thread-portion and the body tissue. More specifically, the portion of the thread which is external of the penetration element is subjected to a larger friction with the tissue compared to the friction between the penetration element itself and the tissue, therefore the withdrawal of the penetration element results in positioning and stretching of the thread within the channel. Any excess of thread, if remains outside of the channel, is then cut. Typically, and following this procedure, the thread remains at the tissue for a relatively long period, until being dissolved.

### SUMMARY

All the above prior art devices are designed for use with threads whose physical properties, such as elasticity, tensile strength, fragility, etc., can sustain the insertion procedure. However, there are threads that have been recently developed, for example, those described in WO 2018/020501, that have certain advantageous properties but may be difficult to successfully deliver intact and sustain during the insertion procedure. These threads, when used with prior art devices, tend to break either during their mounting on the device, or during the insertion procedure.

In some embodiments, a thread-insertion device is provided for use with threads whose one more of their physical properties are not suitable for successful use with prior art devices.

Further, the thread-insertion device is advantageously reliable and simple in structure and operation.

Other objects and advantages of the disclosure will become apparent as the description proceeds.

In some embodiments, a thread-insertion device is provided for creating an intradermal channel in a skin tissue of a patient, with a thread therein, comprising: (a) a main body having a cylindrical-hollowed shape; (b) a penetration element at a distal end of the main body, the penetration element comprising a cannula which is configured to contain the thread in its entirety; and (c) a displacement mechanism, at least a portion thereof permanently contained within the hollow of the main body, the displacement mechanism is configured to effect a relative motion between the cannula and the thread, thereby to expose at least a portion of the thread to a patient's tissue within an intradermal channel. By “permanently,” it is meant during all stages of operation of the device.

In some embodiments, the penetration element is separable from the main body of the device.

In some embodiments, the penetration element is separable from a slidable portion within the main body of the device.

In some embodiments, the penetration element is attachable by means of a luer-lock pair to a distal end of the slidable portion which is contained within the main body.

In some embodiments, the slidable portion is movable along a longitudinal needle between an extended state and a retracted state of the device.

In some embodiments, the connection of the penetration element to the slidable portion is performed when the device is in its extended state, and wherein a transfer of the device to its retractable state fully exposes the thread to the patient's tissue within the intradermal channel.

In some embodiments, the longitudinal needle remains stationary relative to the main body during the transfer of the device to its retractable state, thereby preventing movement of the thread to the proximal direction, and effecting full exposure of the thread to the patient's tissue within the intradermal channel.

In some embodiments, the displacement mechanism is configured to apply a force on a proximal end of the thread, thereby to cause a displacement of the thread such that a length of about 3 mm to about 20 mm of the thread at its distal end protrudes from the distal end of the cannula.

In some embodiments, the displacement mechanism comprises a push button for displacing a needle, wherein the distal end of the needle applies a pushing force on the proximal end of the thread.

In some embodiments, the displacement mechanism comprises a push button for driving a piston, the piston driving a compressed gas within a chamber, and the gas applies a force on the proximal end of the thread.

In some embodiments, the displacement mechanism comprises one or more gears.

In some embodiments, the device further comprising a mechanism for fixing the device in one or more of a loading state and an insertion state.

In some embodiments, one or more of, the entire device, the penetration element and the thread come in a sterile pack.

The disclosure also relates to a method for creating an intradermal channel in a skin tissue of a patient, with a thread therein, the method comprising: (a) providing a device having a main body, the main body having a cylindrical-hollowed shape or lumen; (b) providing a slidable mechanism which is constantly contained within the hollow or lumen; (c) providing a penetration element at the distal end of the device, the penetration element comprising a cannula; (d) providing the thread within the cannula such that the entire length of the thread is occupied within the cannula; (e) creating the intradermal channel using the penetration element; (f) effecting a relative displacement between the thread and the cannula by the slidable mechanism, thereby to expose at least a portion of the thread to the patient's skin tissue within the intradermal channel; and (g) withdrawing the penetration element from the intradermal channel.

In some embodiments, the relative displacement results from a retraction of the cannula relative to the thread.

In an embodiment of the disclosure, the relative displacement results from pushing a proximal end of the thread relative to the cannula, causing the distal end of the thread to protrude a length of about 5 mm to about 10 mm from the distal end of the cannula to within the patient's tissue. In an alternative embodiment, the thread protrudes about 10 mm to about 15 mm from the distal end of the cannula to within the patient's tissue.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIGS. 1a-1e show a device for inserting a thread to within an intradermal channel, according to a first embodiment of the disclosure.

FIGS. 1f-1g illustrate a first variant of the device of the disclosure.

FIGS. 2a-2c show a device for inserting a thread to within an intradermal channel, according to a second embodiment of the disclosure.

FIGS. 3a-3c show a device for inserting a thread to within an intradermal channel, according to a third embodiment of the disclosure.

FIGS. 3d-3f show a second variant of the device of the disclosure.

FIGS. 3g-3h show a third variant of the device of the disclosure.

FIGS. 3i-3j show a fourth variant of the device of the disclosure.

FIG. 3k shows a fifth variant of the device of the disclosure.

FIG. 4a shows a general view of a device for inserting a thread to within an intradermal channel, according to a fourth embodiment of the disclosure.

FIG. 4b shows a cross-sectional view made along plane A-A of the device of FIG. 4a.

FIG. 4c shows another state of the device of FIG. 4b.

FIGS. 4d and 4e show the internal mechanism of the device of FIG. 4a.

FIG. 5a shows a cross sectional view of a device for inserting a thread to within an intradermal channel, according to a fifth embodiment of the disclosure.

FIG. 5b shows another state of the device of FIG. 5a.

FIG. 6a shows a general view of a device for inserting a thread to within an intradermal channel, according to a sixth embodiment of the disclosure.

FIG. 6b is a cross sectional view of the device of FIG. 6a.

FIG. 6c shows the internal mechanism of the device of FIG. 6a.

FIGS. 6d and 6e are cross-sectional enlarge views showing two states of the internal mechanism of the device of FIG. 6a.

FIGS. 7a-7d show several views of a device, according to a seventh embodiment of the disclosure.

FIG. 8a-8c show test results based on the fourth embodiment of the disclosure.

FIG. 9a-9b show test results based on the first embodiment of the disclosure. and

FIG. 10a-10b show test results based on the third embodiment of the disclosure.

### DETAILED DESCRIPTION

As noted, various cosmetic and plastic surgery procedures, for example, a face-lifting procedure, skin rejuvenation, collagen generation, filling, lifting, etc., involve an intradermal insertion and position of a thread inside or underneath the skin of the patient. A variety of devices have been developed throughout the years to carry out this task. However, the prior art devices are designed for use with a relatively high-tensile, flexible, and non-fragile thread, where the thread is mounted within the device's penetration element such that at least a portion (sometimes a large portion) of it is exposed. In some of the prior art devices, the thread has to be significantly bent when mounted on the device, and during the procedure.

Recently, new types of thread have been developed, whose physical properties are not compatible for use with prior art insertion devices. One of such new threads is described, for example, in WO 2018/020501. It has been found that the prior art devices are incapable of successfully completing the insertion task, as the thread tends to break during the mounting of the thread on the device or during the insertion procedure.

The inventors have found that in order to successfully insert a fragile and/or a thread having relatively low tensile strength into a human organ, at least two conditions can lead to a successful delivery: first, during the insertion procedure, the thread should be fully protected within a channel at the penetration element; and second, upon completion of the penetration and the creation of the intradermal channel, it may be sufficient to expose a short portion of the thread (about 3 mm to about 20 mm and in some cases a range of about 5 mm to about 10 mm suffice), to the tissue, and then the withdrawal of the insertion device can be successfully completed while leaving the thread intact within the intradermal channel.

In general, the disclosure provides a device for creating an intradermal channel at a body organ and introducing a thread into the channel. The term “organ,” as used herein, refers mainly to human skin, regardless of any particular location of the skin in the body. Several embodiments and variants for such thread insertion devices are disclosed.

In some embodiments, the device can include a main part (body) and a cannula which is either an integral part of the device or is connectable to the device before the operation to form a part of the device.

In some embodiments, an effective part of the thread can be entirely included within the hollowed cannula and is therefore maintained fully protected during the intradermal insertion process.

After the cannula's insertion (and the thread included therein) is completed, the thread can be exposed to the tissue.

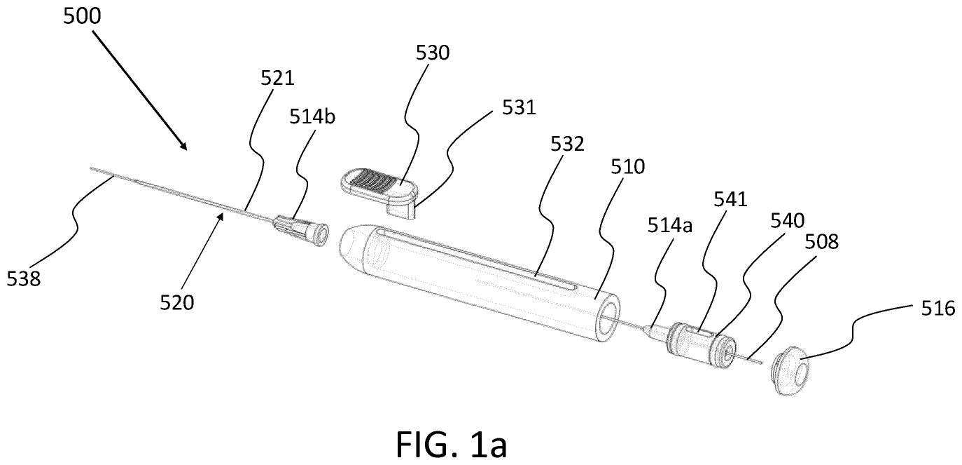

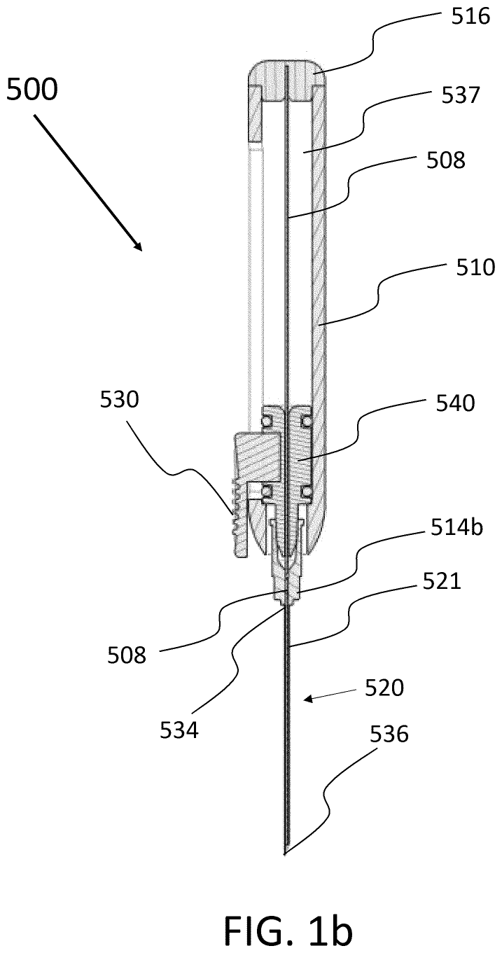

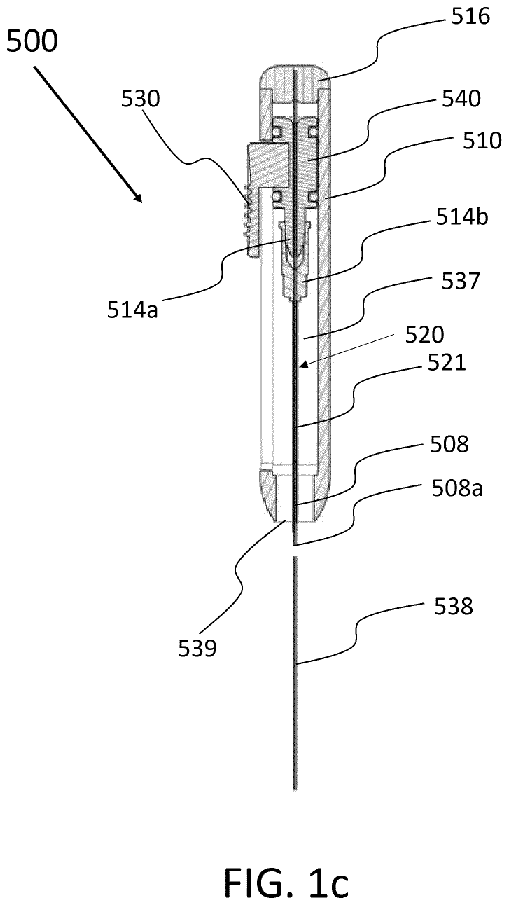

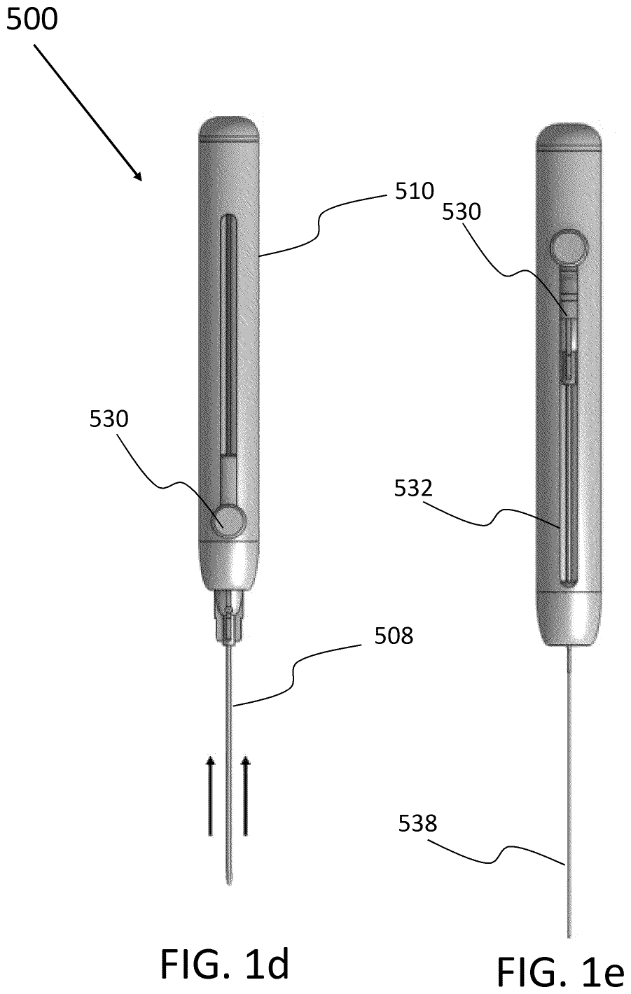

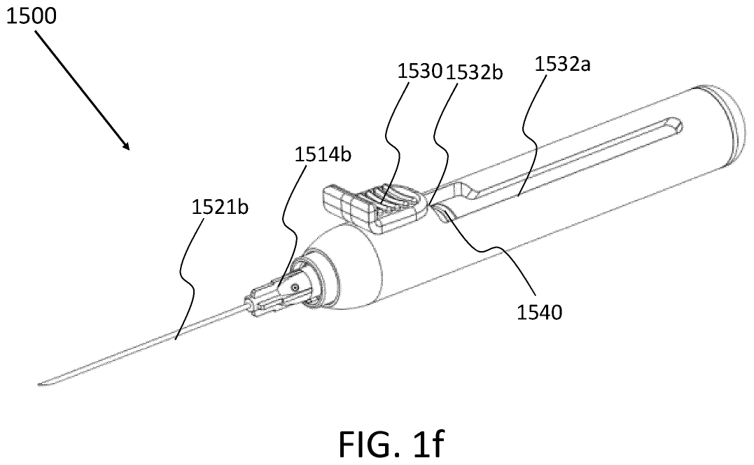

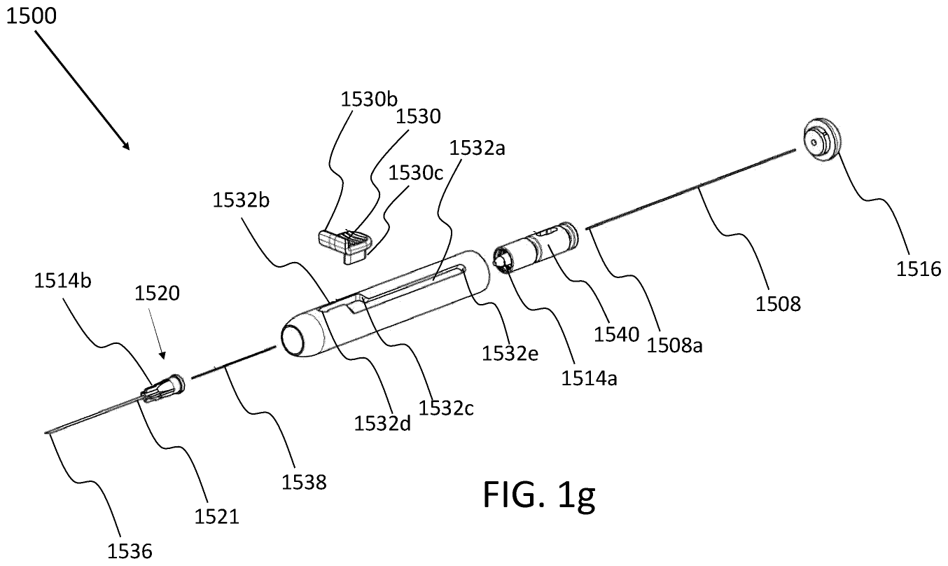

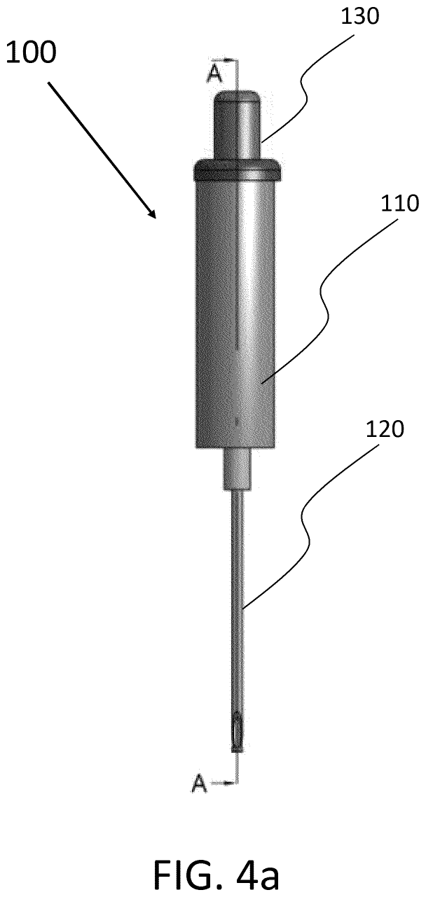

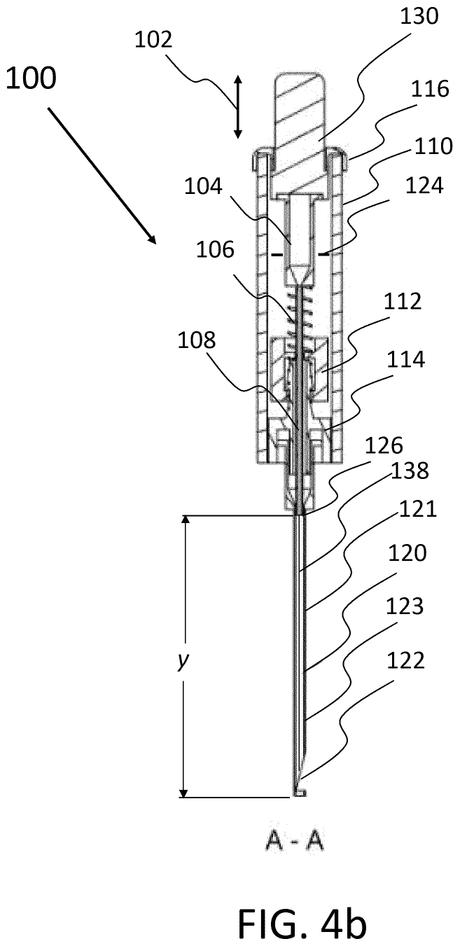

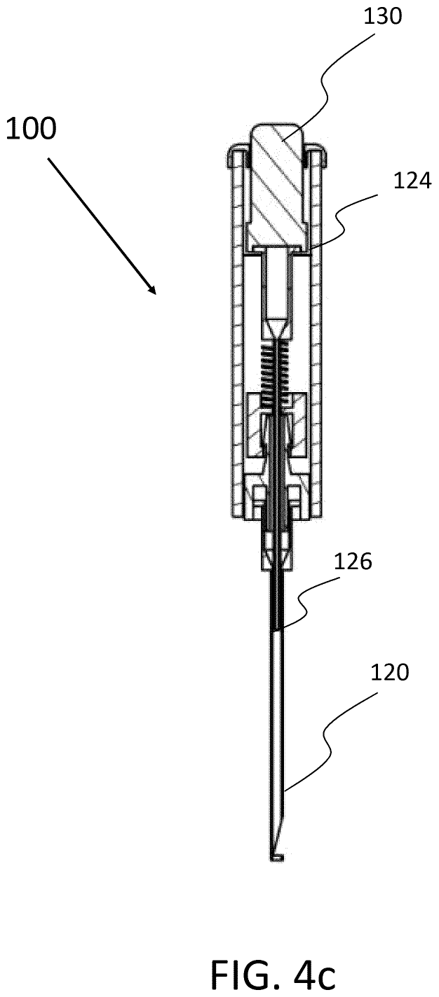

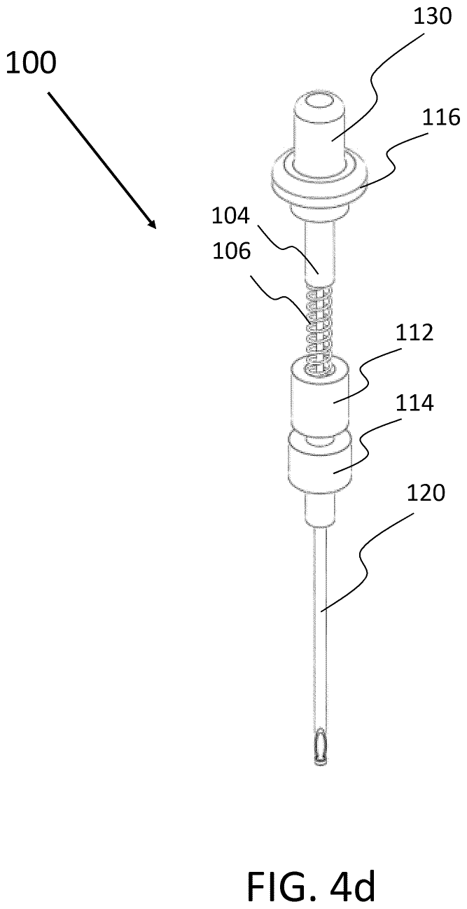

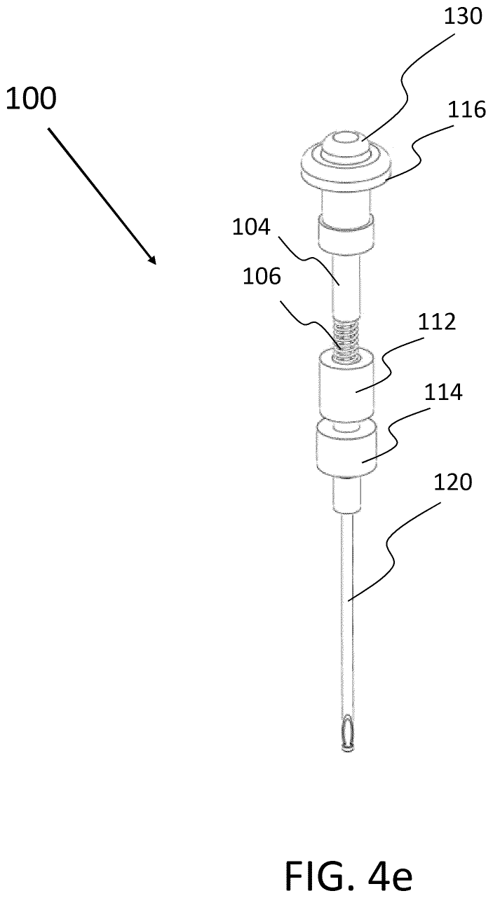

Further, some embodiments of the disclosure can apply two techniques for exposing the thread, once the cannula is inserted, as follows. First, in some embodiments and variants, such as shown in FIGS. 1a to 3k, after the insertion of the cannula (and thread included therein) by the device into the intradermal channel, the user can apply a mechanism that causes a withdrawal of the cannula relative to the thread which remains stationary within the channel. In such a manner, the mechanism exposes substantially all the length of the thread to the tissue. Then, the user pulls the device out of the channel. Second, in some embodiments, such as those shown in FIGS. 4a to 7d, after the insertion of the cannula (and the thread included therein) into the channel, the user can apply a mechanism that pushes the thread forward relative to the cannula, causing the thread to protrude about 3 mm to about 20 mm (in some cases a range of about 5 mm to about 10 mm suffice) of its length from the distal end of the cannula. Then, the user can pull the device (including the cannula) leaving the thread within the channel.

In some embodiments, the device can fully maintain an effective portion of the thread protected within the device during the insertion period (the “effective portion” of the thread is defined as the length of the thread which is occupied or housed within the device, which includes the cannula). When a thread is occupied or housed within the cannula, it is meant that either the thread is entirely housed within the cannula so that no portion of the thread extends outside of or protrudes from a lumen of the cannula or that any portion that protrudes from the cannula is a “non-effective portion.” By “non-effective,” it is meant that such a portion of the thread (i.e., any portion other than the effective portion, which is fully protected within the lumen of the cannula) may protrude from a distal end of the cannula; however, the device's effectiveness with respect to this protruding thread portion is not guaranteed.

FIGS. 1a to 1e show a first embodiment of the device of the disclosure. FIG. 1a shows an exploded view of the device. The device mainly includes: a closure cap 516, a needle 508, a slidable cylinder 540 having: (a) a slot 541; and (b) a male luer-lock portion 514a, a cylindrically shaped main body having (a) space 537; (b) longitudinal slot 532; and (c) distal opening 539 (best shown in FIG. 1c), slidable button 530 having a protrusion 531 (to be engaged with the slidable cylinder 540 at slot 541).

In some embodiments, the device can include a removable (typically disposable) penetration element 520, which includes a hollow cannula 521, having a female luer-lock portion 514b at its proximal end. The penetration element 520 is typically provided in a sterile form, with a thread 538 included within the hollow of cannula 521, where a low friction exists between the thread and the cannula.

When the device is installed, needle 508 elongates substantially along the entire length of the main body 510, and it may even somewhat protrude out of the distal opening 539 as shown in FIG. 1c. More specifically, the distal end of needle 508 may merge with the distal end of the male luer-lock portion 514a, when the slidable cylinder 540 is in its farthest distal position (as shown in FIG. 1B).

The device 500 can have two states: (a) an extended state where the slidable cylinder 540 and the penetration element 520 attached to it are at their farthest distal position (as shown in FIGS. 1b and 1d); and (b) a retracted state where the cylinder 540 and the penetration element 520 attached to it are at their most proximal position (as shown in FIGS. 1c and 1e). The device may optionally be provided with conventional means for locking the slidable cylinder (and therefore also the penetration element) at one or more of the two states. The user uses button 530 to switch the state of the device from one state to the other.

Before the operation, the user brings the device to its extended state (as in FIG. 1b), removes the penetration element 520 (including the thread 538, which is positioned within the hollow of cannula 521) from its sterile pack in which it is contained, and attaches the penetration element 520 to the slidable cylinder 540 employing luer-lock 514. At this stage, the thread is fully contained within cannula 521 and is in fact elongated from tip 534 of luer-lock 514b (where the proximal end of the thread touches the distal end of needle 508) to the sharp distal tip 536 of cannula 521. Next, the user can create a channel at the body organ by inserting the cannula 521 into the patient's organ. Up to this stage, the thread 538 is fully protected within cannula 521. Upon completion of the cannula's insertion, the user transfers the device 500 to its retracted state as shown in FIG. 1c, by pushing button 530 towards the proximal direction. More specifically, during this transfer, cannula 521, which is attached to cylinder 540 (and button 530) is also retracted along needle 508 to the most proximal state shown FIG. 1c. However, while cannula 521 is displaced to its most proximal state, tip 508a of needle 508 prevents a similar movement of thread 538 contained therein, and in such a manner the thread 538 remains stationary, and is fully exposed to the tissue channel. Furthermore, upon completion of the cannula retraction, a small portion of needle 508 can protrude out of the opening 539 device 500, as shown in FIG. 1c. The user completes the procedure by gently pulling the device, thereby removing needle 508 out of the channel. Thread 538 remains fully elongated within the channel.

FIGS. 1f-1g illustrate a variant 1500 (hereinafter, “first variant”) of the device 500 of FIGS. 1a-1e. Similar references in those two groups of figures refer to similar components, the description of which is not repeated herein for brevity but is incorporated here. The device of FIGS. 1a-1e may suffer from two shortcomings, which the device of FIGS. 1f and 1g resolves as follows. First, the installation (namely, “loading”) of cannula 521 by means of the luer-lock pair 514a, 514b involves a force towards the proximal end of the device 500. This force may cause an undesired withdrawal movement of slidable cylinder 540, unless the user applies a counter force on button 530 (in a direction towards the distal end). Moreover, if the installation is performed when the slidable cylinder is not at its absolute distal end (for example, when it is about 10 mm to about 20 mm from the most distal end), the user may face a misalignment problem until the engagement of the luer-lock pair 514a, 514b is completed. The misalignment may cause damage to the needle 508. Second, during the insertion of the penetration element into the organ (i.e., during the creation of the channel within the organ), another withdrawal may occur unless the user applies pressure on button 530, preventing the withdrawal. The first variant can resolve both of the shortcomings.

As can be seen in FIG. 1g, the original longitudinal slot 532 (shown, for example, in FIG. 1a) is divided in the first variant 1500 into two peripherally displaced slots, a main-slot 1532a, and a sub-slot 1532b. Furthermore, the flat button 530 now includes in the first variant an erected front 1530b, enabling the user to more easily apply a proximal-direction force on button 1530. Before the operation, the user brings the device to its extended state, i.e., where button 1530 and the slidable cylinder 1540 attached to it are brought to the most distal end 1532d of sub-slot 1532b. The user removes the penetration element 1520 (including the thread 1538 which is positioned within the hollow of cannula 1521) from its sterile pack in which it is contained. Then, the user attaches (while still forcibly holding button 1530b at the most distal end 1532d) the penetration element 1520 to the slidable cylinder 1540 by means of luer-lock pair 1514a, 1514b. At this stage, the thread is fully contained within cannula 1521 and is in fact elongated from tip 1534 (not shown, see 534 in FIG. 1b) of luer-lock 1514b (where the proximal end of the thread touches the distal end of needle 1508) to the sharp distal tip 1536 of cannula 1521. Next, the user brings button 1530 up to a point where the proximal end 1530c of button 1530 is stopped by the proximal end 1532c of slot 1532b. At this stage, the user creates a channel at the body organ by inserting the cannula 1521 into the patient's organ. The insertion can be performed without the user's finger's engagement on button 1530, thanks to stoppage 1532e engaging end of button 1530c of button 1530. This stoppage releases the user from worrying about a too-early withdrawal of the slidable cylinder 1540. Up to this stage, thread 1538 is fully protected within cannula 1521. Upon completion of the cannula's insertion, the user brings button 1530 to within the main slot 1532a and transfers the device 1500 to its retracted state, i.e., up to stoppage 1532e, by pushing button 1530 towards the proximal direction. More specifically, during this transfer, cannula 1521, attached to cylinder 1540 (and button 1530) is also retracted along needle 1508 to the most proximal position 1532e. However, while cannula 1521 is displaced to its most proximal position, tip 1508a of needle 1508 prevents a similar movement of thread 1538 contained therein, and in such a manner, the thread 1538 remains stationary and is fully exposed to the tissue channel. The user completes the procedure by gently pulling the device, thereby removing needle 1508 out of the channel. Thread 1538 remains fully elongated within the channel. As shown, the first variant 1500 resolves the above two shortcomings (a) and (b) of the device 500 of FIGS. 1a-1e.

The device and procedure of the disclosure, as described, enable the insertion of a relatively rigid thread into a channel at a body organ. The disclosure protects the thread during the procedure and ensures that at the end of the procedure, the thread will remain complete (i.e., unbroken) within the channel.

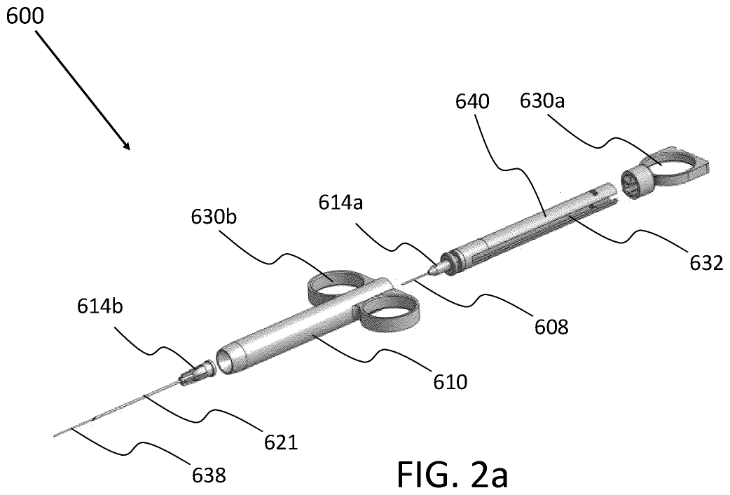

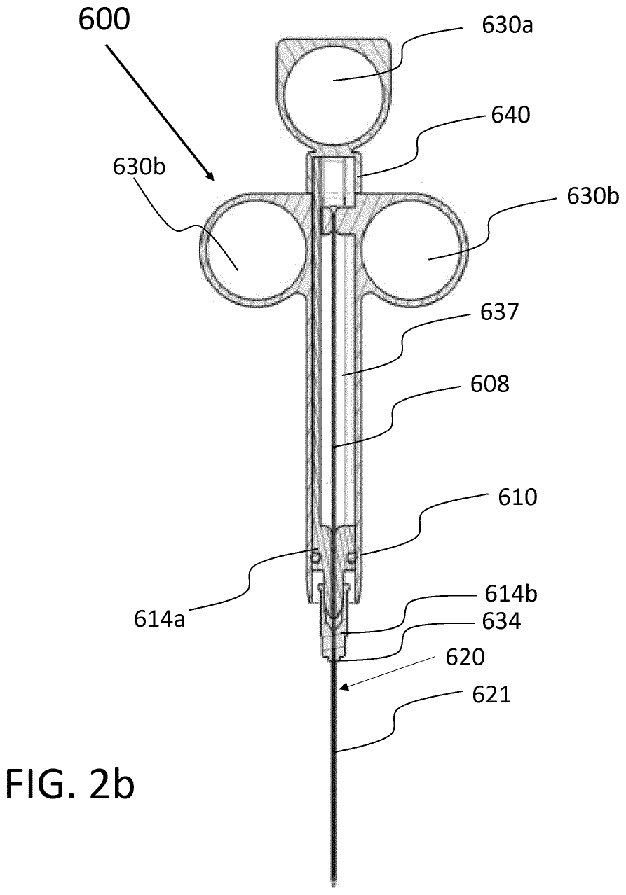

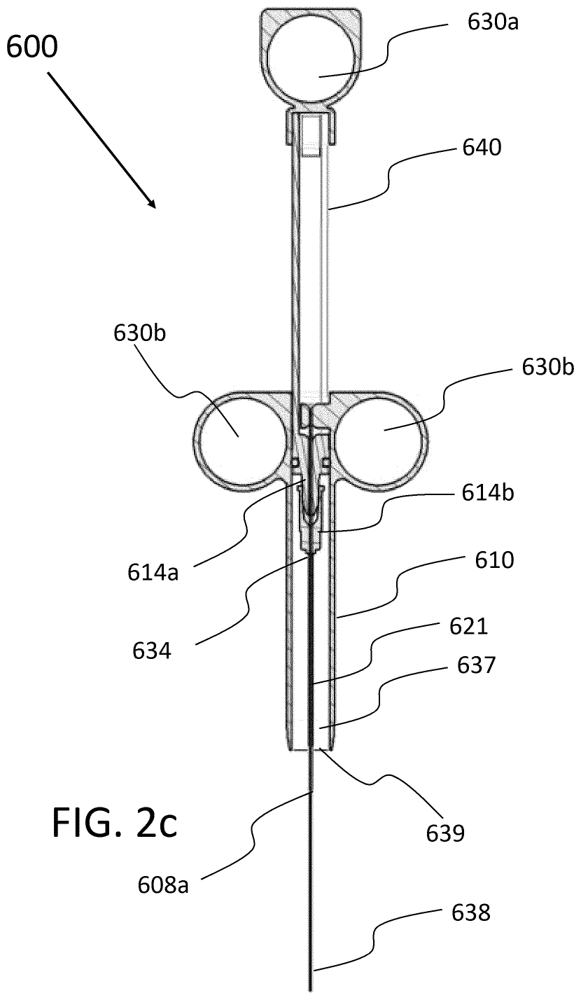

FIGS. 2a-2c show a device 600 according to a second embodiment of the disclosure. FIG. 2a shows the device in an exploded view, FIG. 2b shows the device in its extended state, and FIG. 2c shows the device in its retracted state. The structure of device 600 (of the second embodiment) is somewhat similar to the structure of device 500 (of the first embodiment), and components having similar functionalities to those of the first embodiment have a similar reference number, the description of which is not repeated herein for brevity but is incorporated here.

The device 600 may include the following components: a cylindrically shaped main body 610 having (a) space 637; (b) two-finger units 630 that are attached to the main body 610; and (c) distal opening 639 (best shown in FIG. 2c). The device further includes within the main body 610: a needle 608, and a cylinder 640, which is slidable within the main body over needle 608. Slidable cylinder 640 has: (a) two parallel slots 632; (b) a male luer-lock portion 614a which is attached to the distal end of the slidable cylinder; and (c) a toe unit 630a which is attached to the proximal end of the slidable cylinder 640. The device further includes a removable (typically disposable) penetration element 620, which includes a hollow cannula 621 having a female luer-lock portion 614b at its proximal end. The penetration element 620 is typically provided to the user in a sterile form, with a thread 638 included within the hollow of cannula 621, where low friction exists between the thread and the cannula. The hollow cannula has a sharp tip 636 to enable easy penetration to the patient's organ.

Before the operation, the user brings the device to its extended state (as in FIG. 2b), removes the penetration element 620 (including the thread 638, which is positioned within the hollow of cannula 621) from its sterile pack in which it is contained, and attaches the penetration element 620 to the slidable cylinder 640 by means of luer-lock 614. At this stage, thread 638 is fully contained within cannula 621, and is in fact elongated from tip 634 of luer-lock 514b (where the proximal end of the thread touches the distal end of needle 608) to the sharp distal tip 636 of cannula 621. Next, the user creates a channel at the body organ by inserting the cannula 621 into the patient's organ. Up to this stage, the thread 638 is fully protected within cannula 621. Upon completion of the cannula insertion, the user transfers the device 600 to its retracted state as shown in FIG. 2c, by pulling toe unit 630a towards the proximal direction while holding the main unit 610 stationary (as well as needle 608) by means of the two-finger units 630b. More specifically, during this state transfer, cannula 621, which is attached to cylinder 640 is also retracted along needle 608 to the most proximal retracted state shown FIG. 2c. However, while cannula 621 is displaced to its most proximal state (while being attached to the slidable cylinder 640), tip 608a of needle 608 prevents a similar movement of thread 638 contained therein, and in such a manner the thread 638 remains stationary, becoming entirely exposed to the tissue channel. Furthermore, upon completion of the cannula retraction, a small portion of needle 608 can protrude out of the opening 639 of device 600, as shown in FIG. 1c. The user completes the procedure by gently pulling the device, thereby removing the remaining of needle 608 out of the channel. Thread 638 remains in its entirety elongated within the channel.

The second embodiment of the device and the procedure associated with it, as described, enable the insertion of a thread into a channel at a body organ. The disclosure protects the thread during the procedure and ensures that the thread remains complete at the end of the procedure (i.e., unbroken) within the channel.

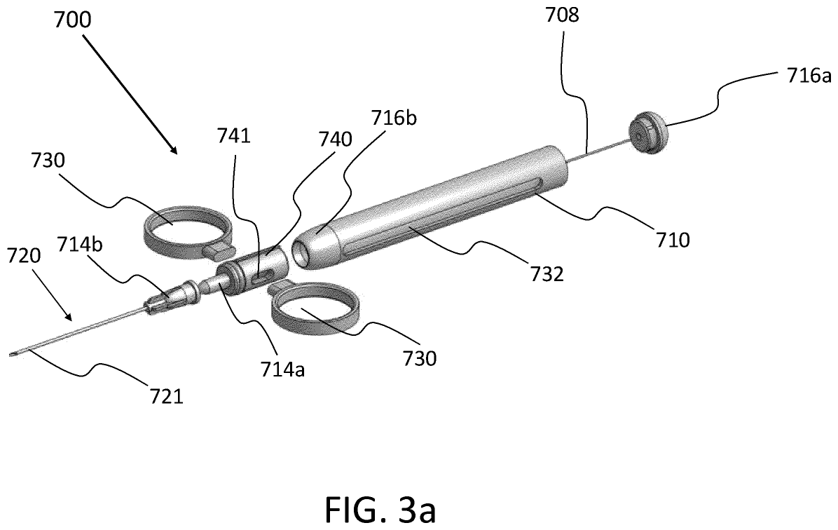

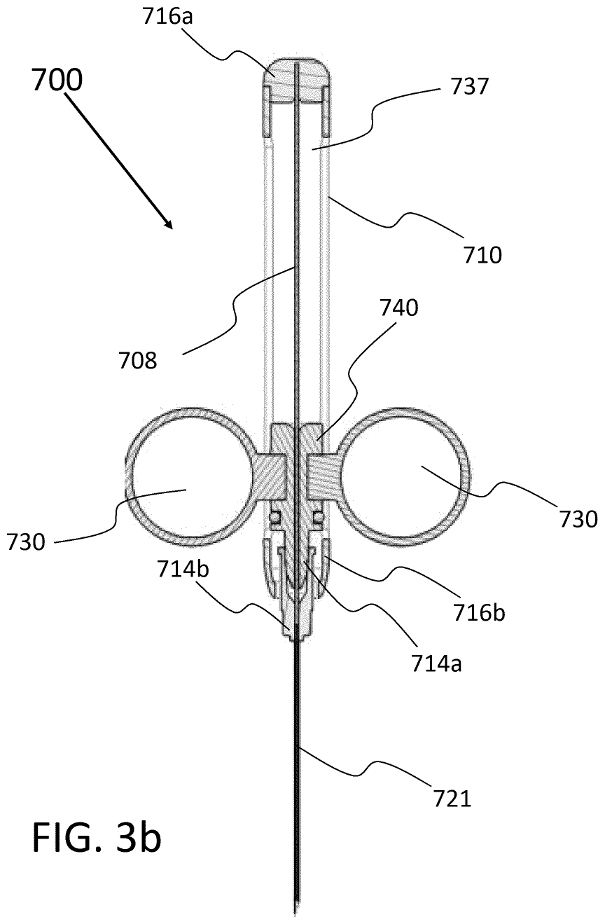

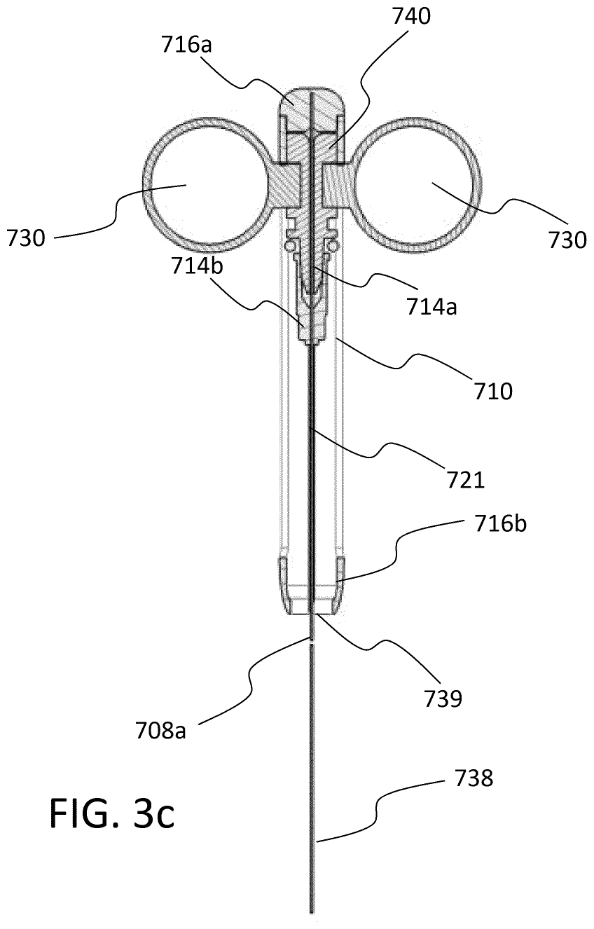

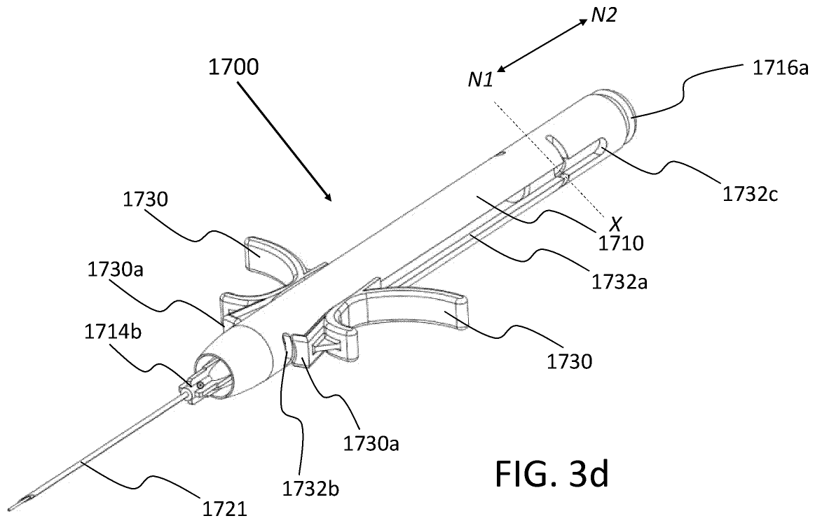

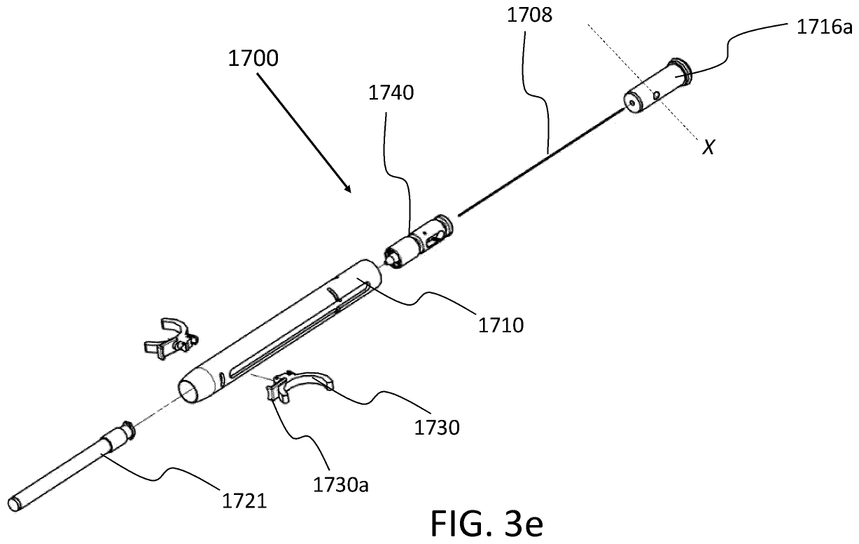

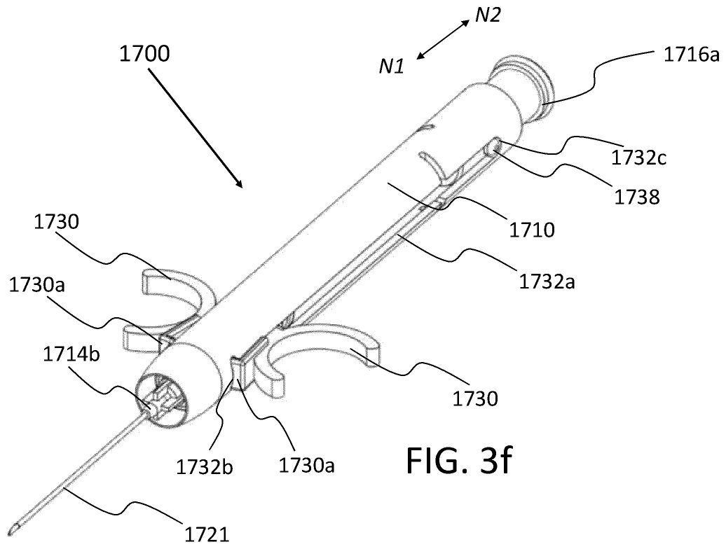

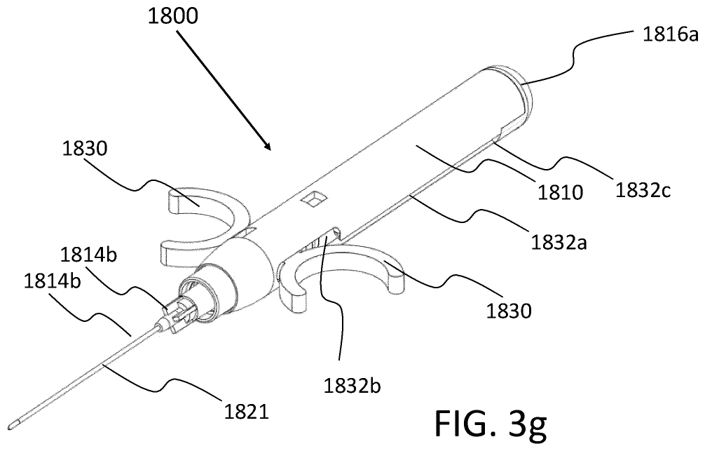

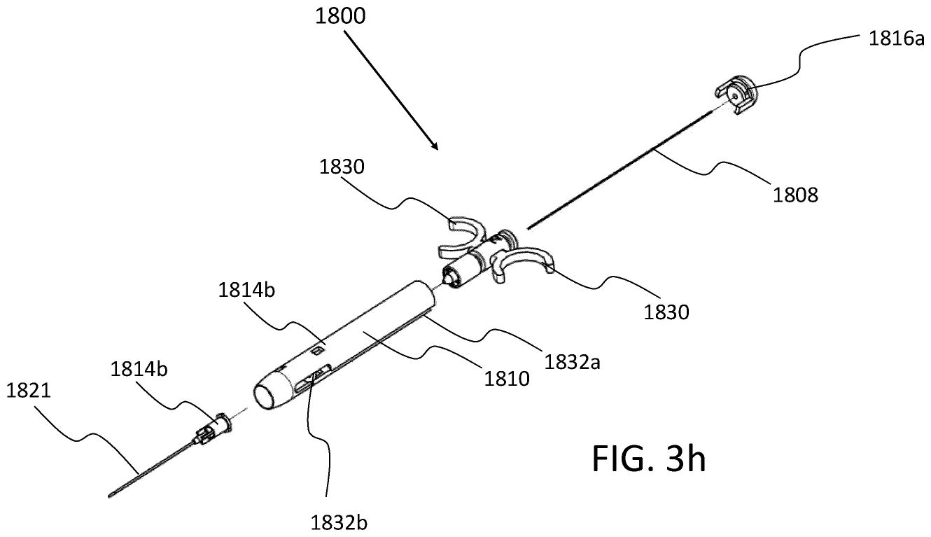

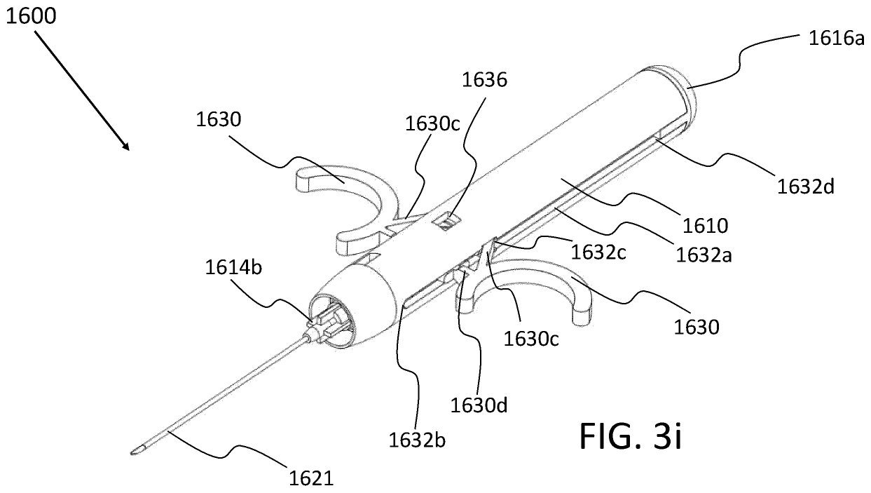

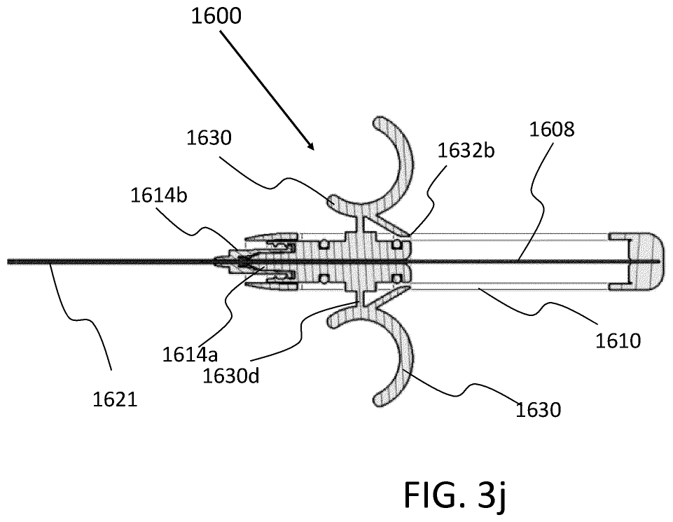

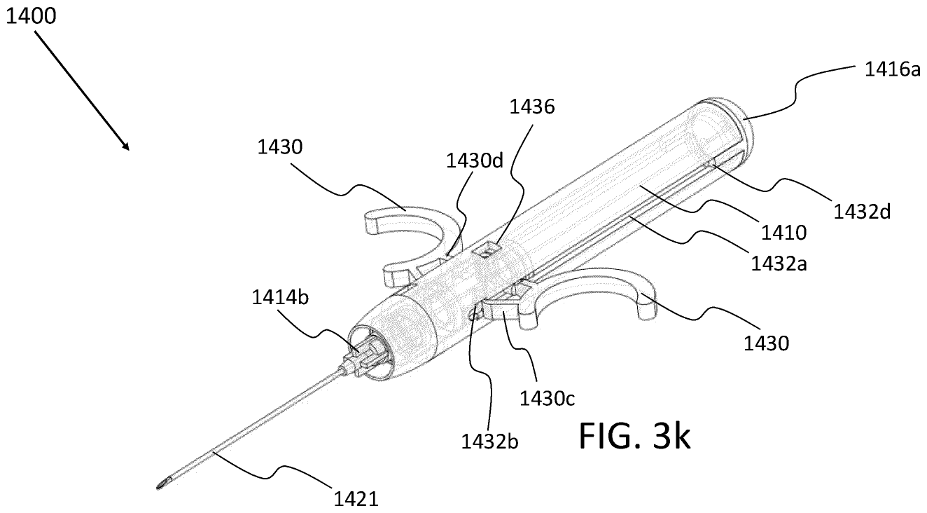

FIGS. 3a-3c show a device 700 according to a third embodiment of the disclosure. FIG. 3a shows the device in an exploded view, FIG. 3b shows the device in its extended state, and FIG. 3c shows the device in its retracted state. The structure of device 700 is somewhat similar to the structures of devices 500 and 600 (of the first and second embodiments), and components having similar functionalities to those of the previous embodiments have similar reference numbers, the description of which is not repeated herein for brevity but is incorporated here. The device 700 can include the following components: a cylindrically shaped main body 710 having (a) space 737; (b) a longitudinal slot 732 (shown in FIG. 3a); (c) top and bottom covers 716a and 716b; and (c) a distal opening 739 at cover 716b (best shown in FIG. 3c). The device further includes within the main body 710: a longitudinal needle 708 and a cylinder 740, which is slidable over needle 708 within the main body. The slidable cylinder 740 includes: (a) two side slots 741 for engagement with two-finger units 730, respectively; and (b) a male luer-lock portion 714a which is attached to the distal end of the slidable cylinder. The device further includes a removable (typically disposable) penetration element 720, which includes a hollow cannula 721 having a female luer-lock portion 714b at its proximal end. The penetration element 720 is typically provided to the user in a sterile form, including a thread 738 within the hollow of cannula 721. Low friction exists between the thread and the internal surface of the cannula. The hollow cannula has a sharp tip 736 to enable easy penetration to the patient's organ.

Before the operation, the user brings the device to its extended state (as in FIG. 3b), removes the penetration element 720 (including the thread 738 which is positioned within the hollow of cannula 721) from its sterile pack in which it is contained, and attaches the penetration element 720 to the slidable cylinder 740 by means of luer-lock 714. At this stage, thread 738 is fully contained within cannula 721 and is in fact elongated from tip 734 of luer-lock 714b (where the proximal end of the thread touches the distal end of needle 708) to the sharp distal tip 736 of cannula 721. Next, the user creates a channel at the body organ by pushing the main body 710, and inserting the cannula 721 into the patient's organ. Up to this stage, thread 738 is fully protected within cannula 721. Upon completion of the cannula's insertion, the user transfers the device 700 to its retracted state, as shown in FIG. 3c, by pulling the finger units 730 towards the proximal direction while holding the main body 710 stationary. More specifically, during this state transfer, cannula 721, which is attached to cylinder 740 is displaced along needle 708 to the most proximal retracted state shown FIG. 3c. However, while cannula 721 is displaced to its most proximal state (while being attached to the slidable cylinder 740), tip 708a of needle 708 prevents a similar movement of thread 738 contained therein, and in such a manner the thread 738 remains stationary, becoming entirely exposed to the tissue within the channel. Furthermore, upon completion of the cannula retraction, a small portion of needle 708 can protrude out of the opening 739 of device 700, as shown in FIG. 3c. The user completes the procedure by gently pulling the device, thereby removing the remaining of needle 708 out of the channel. Thread 738 remains in its entirety elongated within the channel.

The third embodiment of the device and the procedure associated therewith, as described, enable the insertion of a thread into a channel at a body organ. The disclosure protects the thread during the procedure and ensures that the thread remains complete at the end of the procedure (i.e., unbroken) within the tissue channel.

FIGS. 3d to 3f show a variant 1700 (hereinafter, “the second variant”) of device 700 of FIGS. 3a-3c. The device 700 may suffer from the same shortcomings (a) and (b) that device 500 suffers from (and which device 1500 resolves). References in FIGS. 3d to 3f that are similar to those of FIGS. 3a-3c refer to similar components, the description of which is not repeated herein for brevity but is incorporated here. The during the loading state of device 1700 (shown in FIG. 3f), the two-finger units 1730a are brought to their most distal position, where the two protrusions 1730a seat within respective sub-slots 1732b. This location locks any movement of finger units 1730, and of the slidable cylinder 1740 (shown in FIG. 3e). At the intermediate stage shown in FIG. 3d, however, needle 1708 is too far to the distal end (not shown), a situation which may result in misalignment of the two luer-locks 1714 (same as shortcoming (a) discussed above with respect to the device 500). To eliminate the shortcoming of misalignment, top cover 1716 is made movable along the device's longitudinal axis between points N1 and N2. More specifically, top cover 1716a is rigidly attached to a proximal section of needle 1708. Before attachment of the cannula 1721 by means of luer-lock 1714, button 1716a is pulled from its normal position N1 (shown in FIG. 3d), about 15 mm to about 20 mm in the distal direction, e.g., from an initial position where pin 1738 (shown in FIG. 3f, omitted in FIG. 3d) is located at a position that coincides with axis x toward the end-of-movement position 1732c (shown in FIG. 3f). This displacement of button 1716a temporarily also pulls the needle 1708 about 15 mm to about 20 mm in the distal direction. This is the loading state where the cannula 1721 is attached to the device. At this loading stage, the sliding cylinder 1740 is locked (by means of protrusions 1732b), and the misalignment is eliminated (by means of displacing the needle 1708 to the distal direction). With the thread included therein, the cannula can be safely attached to the sliding cylinder 1740 by means of the luer-lock pair 1714a, 1714b. Following the cannula's attachment, the user returns button 1716a (and needle 1708) to their N1 position, and the device 1700 is ready for operation. Then, the user may continue by inserting the cannula into the organ, namely, creating a channel. Next, when the cannula is fully positioned within the tissue channel, the user releases protrusions 1730a from sub-slots 1732b and pulls the finger units to the proximal end (along slot 1732a, while simultaneously pushing button 1716a (for example, by the user's thumb) to the distal direction. This operation withdraws the cannula and exposes the thread to the organ within the created channel. Then, the user may gently remove the rest of the cannula from the organ-channel. The thread remains within the channel.

FIGS. 3g and 3h show a third variant 1800 of the disclosure. The third variant combines characteristics from the first and second variants, while components having similar reference signs serve similar functionalities, the description of which is not repeated herein for brevity but is incorporated here. Similar to the first variant, the device 1800 includes a stationary cap 1816a, a stationary needle 1808, two main slots 1832a (the first variant includes one main slot 1530), two sub-slots 1832b (the first variant includes one sub-slot), and other similar components. However, rather than a single button 1530, the device 1800 includes two finger units 1830, similar to finger units 1730 of the second variant. The operation may be substantially the same as the operation of device 1500, mutatis mutandis. To transfer the finger units 1830 from within the sub-slots 1832b to the main slots 1832a, the user has to hold the finger units by one of his hands and to slightly rotate body 1810 (clockwise in FIG. 3g) by his other hand.

FIGS. 3i and 3j show a fourth variant 1600 of the disclosure. The fourth variant combines characteristics from the embodiment 700 of FIG. 3a and from the second variant 1700, while components with similar reference signs serve similar functionalities, the description of which is not repeated herein for brevity but is incorporated here. Similar to the device 700, the device 1600 includes a stationary cap 1616a, a stationary needle 1608, two parallel slots 1632, and other similar components. Initially, and to load the cannula 1621, the two-finger units 1630 are brought to the most extreme distal position 1632b. At this position, a “1” may be displayed within window 1636 to indicated the first step. The user firmly maintains the device at point 1632b, while attaching the cannula 1621 by means of luer lock pair 1614a, 1614b. Next, following of the attachment of cannula 1621, the user brings the finger units to an intermediate location, where the two first arms 1630c seat within two parallelly-positioned slots 1632c. A “2” may be displayed within window 1636 at this fixed position to indicated a second step. While in this intermediate fixed position, the device is ready for insertion into the patient's organ, while the user can release his fingers from finger units 1630. Upon completing the insertion of cannula 1621 into the tissue channel, the user can release the first arms 1630c from slots 1632c (by slightly tilting units 1630 about second arms 1630d) to pull finger units 1630 to their final position 1632d. The pulling of units 1630 withdraws the cannula 1621 from the channel and exposes the thread to the organ.

FIG. 3k shows a fifth variant 1400 of the device of the disclosure. The fifth variant is similar to the fourth variant 1600, while components having similar reference signs serve similar functionalities, the description of which is not repeated herein for brevity but is incorporated here. In device 1400, the intermediate position 1632c (of the fourth variant) is eliminated. Position 1432b becomes both the loading position and the position where the insertion into the organ is performed. Initially, finger units 1430 are brought to most distal position 1432b, where arm 1430c rests in a fixed position within sub-slot 1432b. While at a fixed position, the cannula 1421 is attached to the device. Next, while still in the fixed position, the cannula is inserted into the organ, creating a channel. Upon completion of the insertion of cannula 1421 into the channel, the user can release the first arms 1430c from slots 1432b (by slightly tilting units 1630 about second arms 1430d), and pull finger units 1430 to their final position 1432d. The pulling of units 1430 withdraws the cannula 1421 from the channel and exposes the thread to the organ.

FIG. 4a shows a device 100 for the insertion of a thread into an intradermal channel within a body organ, according to a fourth embodiment of the disclosure. FIG. 4b shows a cross-sectional view made along plane A-A of the device 100. The device 100 mainly comprises a main body 110, a penetration unit 120, and a push-button 130. As shown in FIG. 4b, the penetration unit 120 includes a penetration element 121 in the form of a cannula which is adapted to contain the thread 138 fully. The penetration element (cannula) 121 has a sharp tip 122 at the distal end, enabling it to penetrate into the organ while creating a channel within the tissue. The device's internal mechanism further includes a push button 130 at the proximal end of the device, with a piston 104 attached thereto, and a needle 108 attached to the piston. In the stationary state of the device, the combined unit which consists of the button 130, piston 104, and needle 108 is pushed towards the proximal direction by means of spring 106. Spring 106 is maintained at its distal end within a recess at adaptor 112. Cap 116 locks the internal structure to the main body 110, as shown. The distal end of needle 108 is somewhat occupied within a hollow channel 123 of the penetration element 121. The penetration unit 120 may be made removable, and if so, it is connected to the main body 110 by means of a luer-lock connection 114. The device also has a stoppage 124 for defining a bottom end-of-displacement for button 130, when the button is pressed.

As was noted, the thread which is used by the device of the disclosure may be incompatible for use by prior art devices due to one or more physical properties. For the sake of sterilization, the thread should preferably be provided in one of two alternatives: (a) within a penetration unit 120, which is separate from the main body 110; or (b) within the penetration unit 120, which is already connected to the main body 110. More specifically, the length of the thread should be substantially, which is the length between the distal end of needle 108 and the distal tip 122 of the penetration element 120, such that in the initial state, the thread is occupied in its entirety within the channel 123 of the penetration element 121, but does not protrude through the distal tip 122. The thread diameter should be made somewhat less than the diameter of channel 123 (namely, the internal diameter of the cannula) to ensure minimum friction between the thread and the cannula's internal wall. For example, the difference between the diameters may be larger than 10%. This requirement is valid for all the embodiments and variants of the disclosure.

Next, the medical practitioner inserts the penetration element 121 into the patient's organ at the desired location, and by the insertion creates an intradermal channel. Following the channel creation, the medical practitioner activates the device by pushing button 130 and piston 104 (against the force of spring 106) up to the stoppage point 124. Piston 104, in turn, pushes the needle 108, which may be rigidly attached to it further within the hollow channel 123 of the penetration element 120. This state of device 100 is shown in FIG. 4c. The distal end 126 of needle 108 therefore pushes the proximal end of the relatively rigid thread, which in turn somewhat protrudes at its distal end outside of the distal tip 122 of the penetration element 120. In total, push-button 130 is displaced about 3 mm to about 20 mm (in some cases about 5 mm to about 10 mm suffice) from its initial position of FIG. 4b up to the stoppage point 124 of FIG. 4c. The displacement of about 3 mm to about 20 mm has been found to provide a sufficient thread-protrusion length which ensures, on the one hand, non-breakage of the thread, and on the other hand, sufficient friction between the protrusion of the thread and the body tissue such that the thread remains within the created tissue channel upon withdrawal of the penetration element 121. FIGS. 4d and 4e show the internal mechanism of device 100, in its stationary state and in its pressed state, respectively.

Then, a withdrawal by the medical practitioner of the penetration element from the intradermal tissue channel leaves the thread stretched within the channel. This is based on the observation that the friction between the exposed portion of the thread and the tissue is larger than the friction between the non-exposed portion and the internal wall of channel 123 of the penetration element.

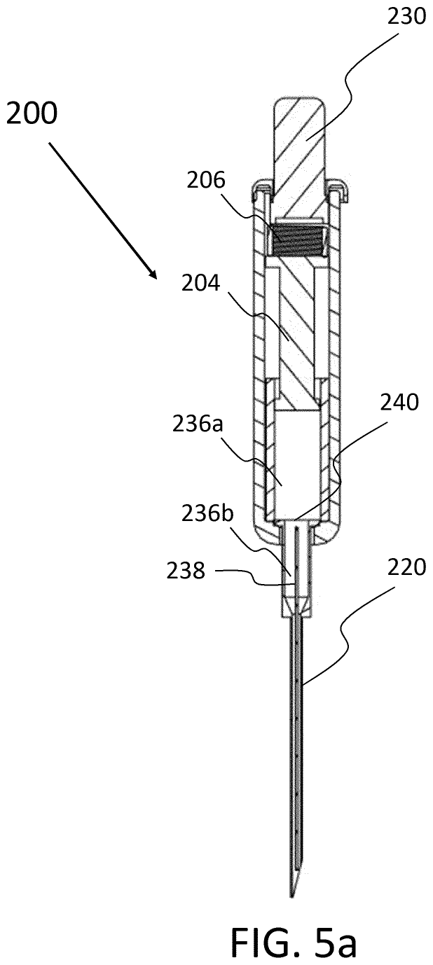

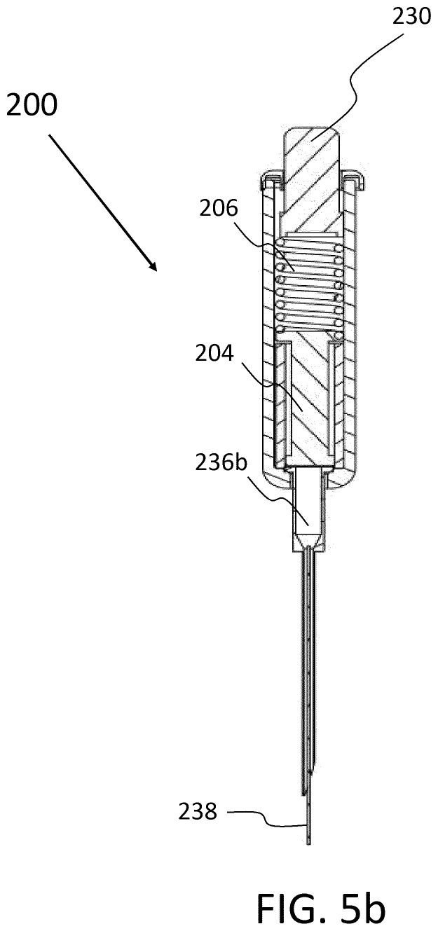

FIGS. 5a and 5b are general cross-sectional views of a gas-operated device 200 according to a fifth embodiment of the disclosure. The gas-operated device is shown in FIG. 5a in its initial state, and in FIG. 5b in its activated state. The device basically includes a press-button 230, piston 204, spring 206, a penetration unit 220, and a gas chamber 236 (which is divided into a primary chamber 236a and a secondary chamber 236b, separated by a membrane 240 to prevent gas transfer between them in the initial state). In its initial state shown in FIG. 5a, button 230 is in a proximal location, spring 206 is locked in a compressed state, and piston 204 is also maintained in a proximal location. The relatively rigid thread 238 is fully occupied within the device as shown.

The activated state of the device is shown in FIG. 5b. Following the creation of an intradermal tissue channel by inserting the penetration element into the body organ, the user pushes button 230 into its distal state, which in turn releases and transfers spring 206 into a non-compressed state, as shown. The spring, in turn, pushes piston 204 to its distal state, which in turn pushes the compressed air within chamber 236a into chamber 236b (by breakage of the membrane 240), which in turn causes a displacement in the order of about 3 mm to about 20 mm of the thread 238 to the distal direction. This displacement causes the thread to protrude about 3 mm to about 20 mm from the distal end 122 of the protrusion element. The rest of the device's structure and operation is similar to the ones described before, mutatis mutandis.

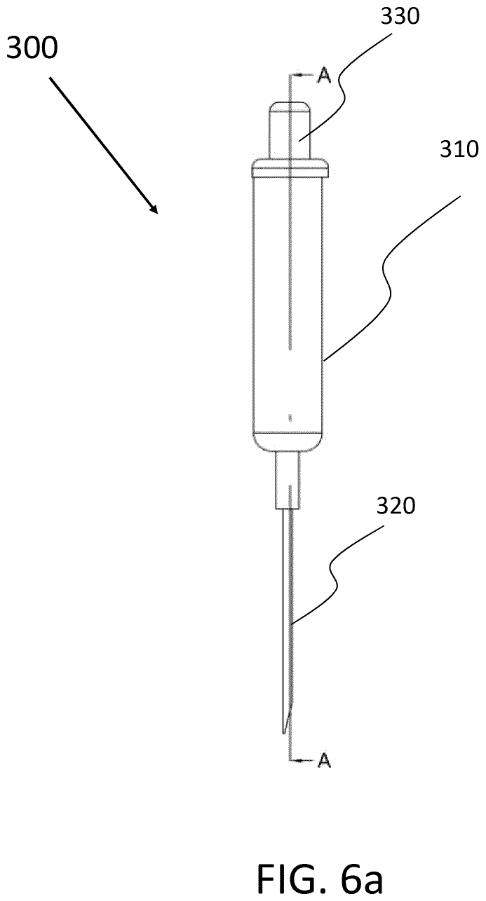

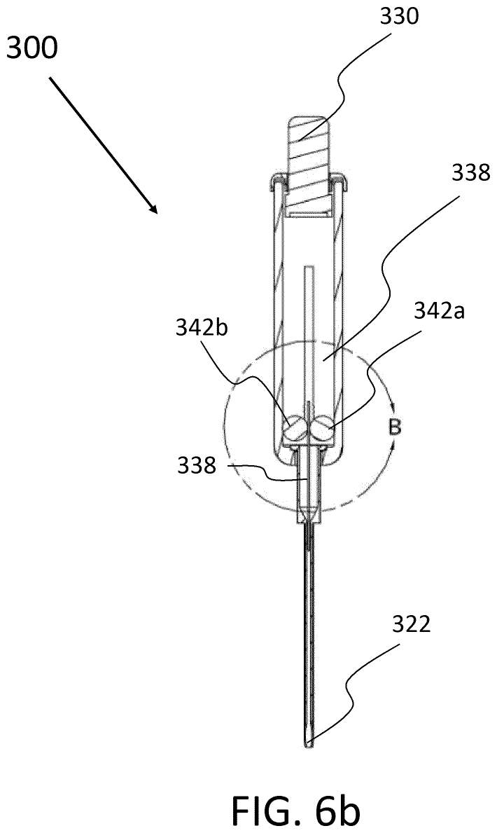

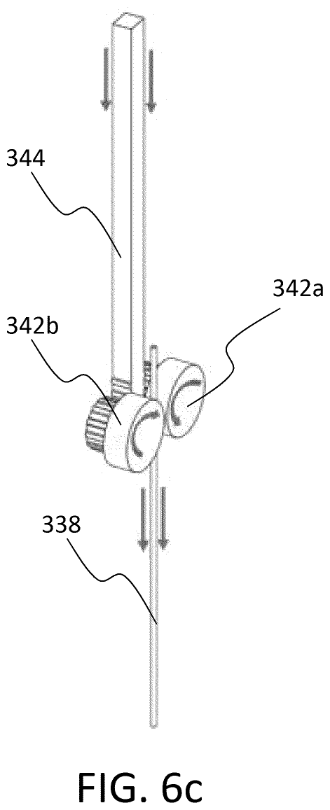

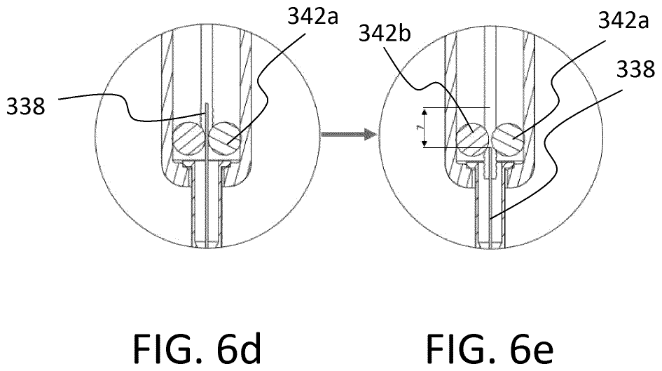

FIG. 6a shows a general view of an exemplary device 300, according to a sixth embodiment of the disclosure. FIG. 6b shows a cross-sectional view of device 300. FIG. 6c shows the main mechanism of device 300, FIG. 6d shows the device's mechanism in its initial state, and FIG. 6e shows the mechanism of the device in its activated state. As best shown in FIG. 6c, the device 300 includes a gear mechanism for pushing the distal end of the relatively rigid thread 338. The mechanism includes two gear wheels, 342a and 342b, and a central driving gear rod 344. The driving gear rod 344 is pushed (activated) by means of push-button 330. A displacement of rod 344 causes a respective turn of the two wheels 342a, 342b, that in turn push the thread 338, resulting in a respective displacement of the thread. Again, the displacement, as well as the protrusion through edge 322 are limited to about 3 mm to about 20 mm (about 7 mm is indicated in the example of FIG. 6d).

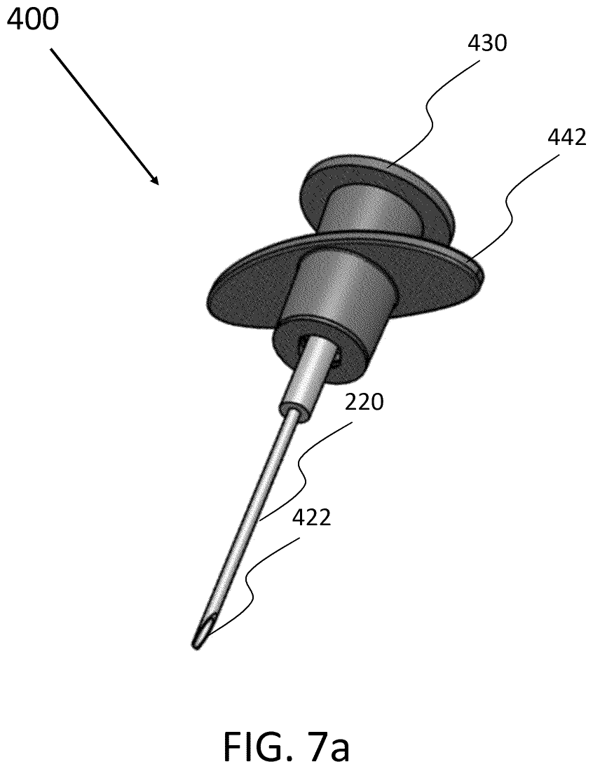

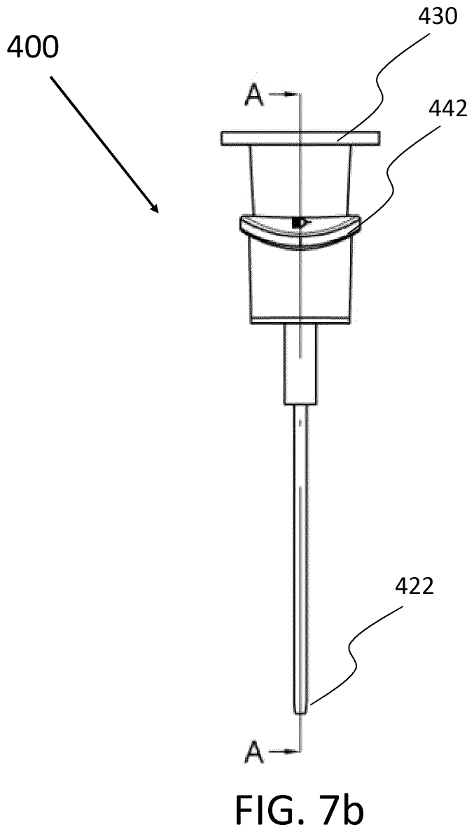

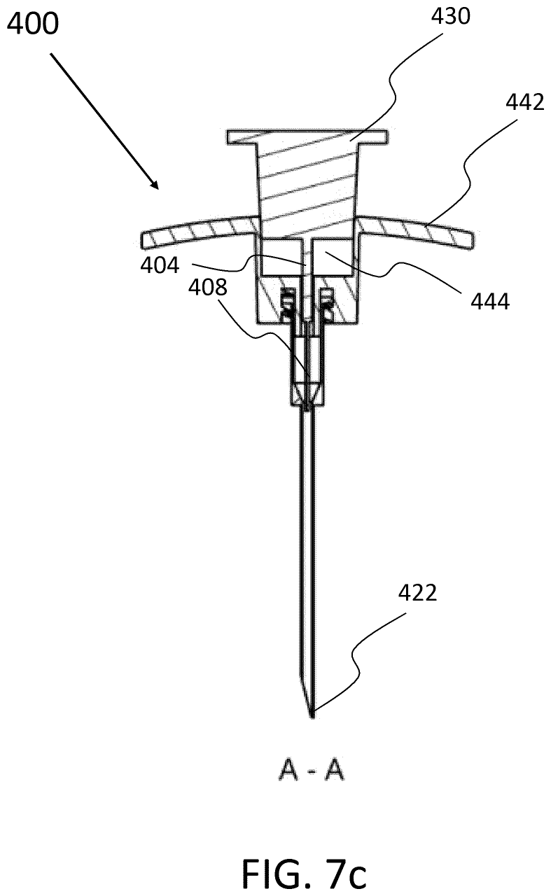

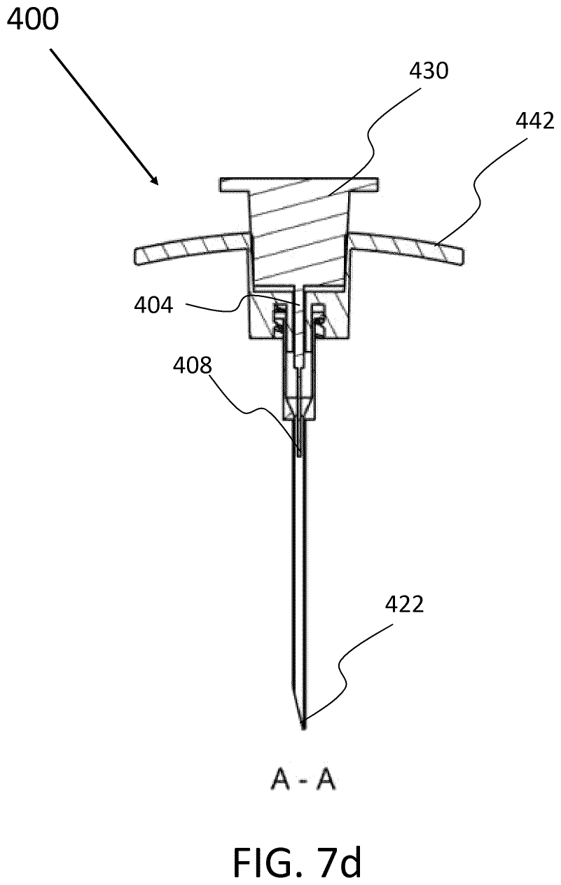

FIG. 7a shows a general view of a device 400, according to a seventh embodiment of the disclosure. FIG. 7b shows a side view of device 400. FIG. 7c is a cross-sectional view showing the device 400 in its initial state, and FIG. 7d is a cross-sectional view showing the device 400 in its activated state. The device 400 is somewhat similar to the device 100 of FIGS. 7a-7e, while components having similar reference signs serve a similar functionality. The device 400 basically includes a push-button 430, having at its distal end a piston 404. Similar to the device 100, the distal end of piston 404 is in contact with a proximal end of movable needle 408, whose distal end is in contact with the proximal end of a relatively rigid thread (not shown). When activated, push-button 430 moves down, causing a displacement of about 3 mm to about 20 mm of needle 408. Needle 408, at its distal end, similarly pushes the thread (not shown) to protrude about 3 mm to about 20 mm from edge 422. The operation of device 422 is based on a friction between the external wall of the push button 430 and the internal wall of chamber 444 (FIG. 7c), rather than on a spring, such as spring 106 of device 100. The device further includes two ginger grippers 442 for ease of operation.

The level of thread rigidity that is used in the context of this disclosure may be determined experimentally. For the fourth, fifth, and sixth embodiments, any thread that the device of the disclosure can successfully push to a protrusion depth (from the tip of the cannula) of about 3 mm to about 20 mm (in some cases a range of about 5 mm to about 10 mm suffice) within the tissue channel without breakage, is suitable.

### EXAMPLES

Three embodiments of the disclosure were tested using threads composed of Hyaluronic acid and Calcium hydroxyapatite prepared according to WO 2018/020501. For each insertion-device prototype, a thread having an average diameter of 376.5 μm and a length of about 43 mm was used. The threads were fully inserted to 21G×2″ needles attached to the device and injected using the disclosure's insertion devices to different models, i.e., artificial skin (deep suture pads by SimSkin), 20% gelatin-based gel, etc. imitating a human skin.

Embodiments 1, 3, and 4 were tested. At each test, the thread was completely extruded from the device and remained intact within the model.

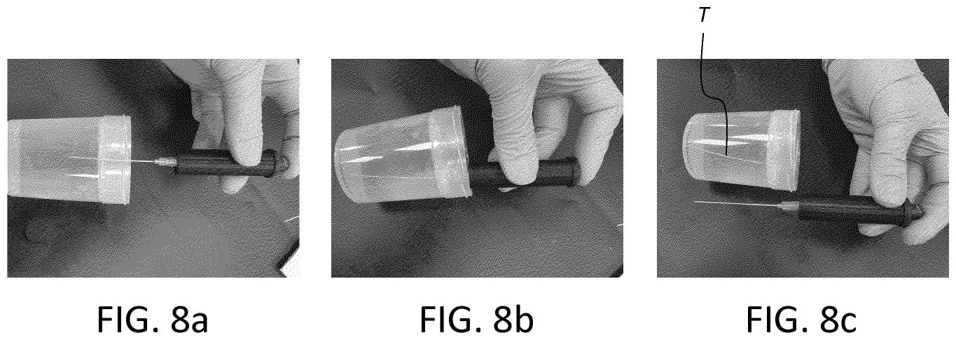

FIGS. 8a-8c show insertion of a thread T by the device of the fourth embodiment. FIG. 8c shows how the thread T is intact within the model after completing the tested procedure.

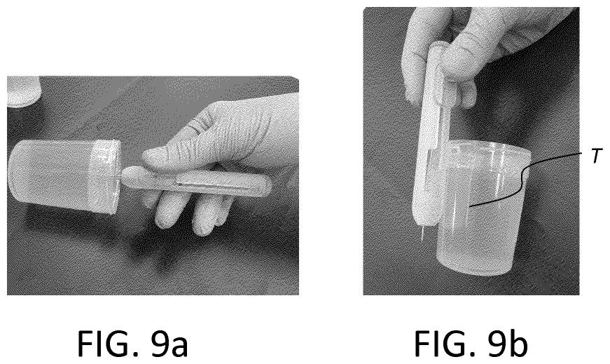

FIGS. 9a-9b show insertion of a thread T by the device of the first embodiment. FIG. 9b shows how the thread T is intact within the model after completing the tested procedure.

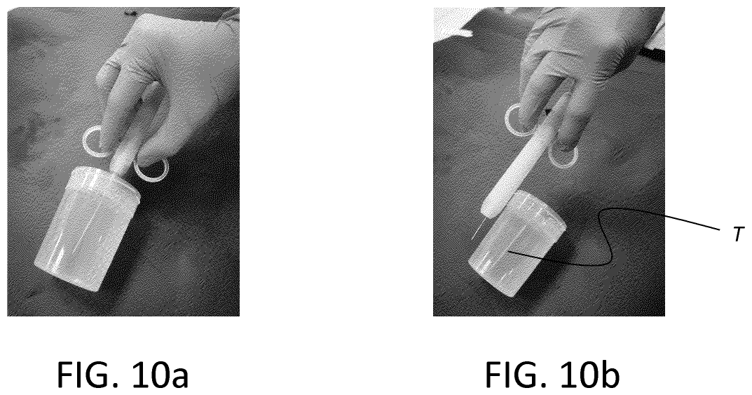

FIGS. 10a-10b show insertion of a thread T by the device of the third embodiment. FIG. 10b shows how the thread T is intact within the model after completing the tested procedure.

Additional insertion tests were performed with prototypes of embodiment 1500 and 1700. The insertions were performed with 44 mm threads inserted manually into 21G×2″/0.80×50 mm (B. BRAUN, 4665503) needles. In all the tests, the difference between the thread's diameter and the internal diameter of the cannula was larger than 10%. The insertions were divided into two stages. The first stage was performed in a Benchtop Testing on different mediums, and in the second stage, the insertions were performed on 7 patients by experienced doctors.

TABLE 1
threads insertions to in-vitro mediums

| | | Number of | Success rate |
|---|---|---|---|
| | Embodiment# | thread | of thread |
| Medium Type | Prototype | insertions | insertions* |
| Artificial skin** (SimSkin) | 1500 | 14 | 100% |
| | 1700 | 14 | |
| Blue sponge | 1500 | 10 | 100% |
| | 1700 | 19 | |
| Ballistic gel (40% Gelatin), | 1500 | 4 | 100% |
| Sigma Aldrich, G7041) | 1700 | 4 | |
| Ballistic gel (20% Gelatin), | 1500 | 6 | 100% |
| Sigma Aldrich, G7041) | 1700 | 6 | |

*A successful insertion is a case when a whole thread is inserted and remains in place below the surface with no protruding edges or tears following the insertion.
**Deep suture pads- SimSkin

TABLE 2
Clinical tests insertions

| | | Number of | Success rate |
|---|---|---|---|
| | Insertion device | threads | of thread |
| Patient number | Prototype | insertions | insertions* |
| Patients 1 | 1500 | 15 | 100% |
| | 1700 | 7 | |
| Patients 2 | 1500 | 18 | 100% |
| | 1700 | 18 | |
| Patients 3 | 1700 | 32 | 100% |
| Patients 4 | 1500 | 15 | 100% |
| Patients 5 | 1700 | 24 | 100% |
| Patients 6 | 1500 | 15 | 100% |
| Patients 7 | 1700 | 20 | 100% |

*A successful insertion is a case when a whole thread is inserted and remains in place below the surface with no protruding edges or tears following the insertion.

It should be noted that many tries to completely introduce the same thread by typical prior art devices (for example, those commonly called “needles”) were failed.

As shown, the disclosure provides a device for creating, by means of a cannula, an intradermal channel at a human organ, which follows by the introduction of a thread to within the channel. The four embodiments of the disclosure are characterized by the inclusion of a mechanism that is configured to push the thread from its proximal end to a final location where its distal end protrudes about 3 mm to about 20 mm from an edge of the cannula of the device. As noted, it has been found that this size of protrusion is sufficient.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1 or clause 20. The other clauses can be presented in a similar manner.

Clause 1. A device for creating an intradermal channel in a skin tissue of a patient with a thread therein, the device comprising: a main body having a cylindrical-hollowed shape; a penetration element at a distal end of the main body, the penetration element comprising a cannula which is configured to contain the thread in its entirety; and a displacement mechanism, at least a portion thereof permanently contained within the hollow of the main body, the displacement mechanism is configured to effect a relative motion between the cannula and the thread, thereby to expose at least a portion of the thread to a patient's tissue within an intradermal channel.

Clause 2. A device according to Clause 1, wherein the penetration element is separable from the main body of the device.

Clause 3. A device according to Clause 1, wherein the penetration element is separable from a slidable portion within the main body of the device.

Clause 4. A device according to Clause 3, wherein the penetration element is attachable by means of a luer-lock pair to a distal end of the slidable portion which is contained within the main body.

Clause 5. A device according to Clause 4, wherein the slidable portion is movable along a longitudinal needle between an extended state and a retracted state of the device.

Clause 6. A device according to Clause 5, wherein the connection of the penetration element to the slidable portion is performed when the device is in its extended state, and wherein a transfer of the device to its retractable state fully exposes the thread to the patient's tissue within the intradermal channel.

Clause 7. A device according to Clause 6, wherein the longitudinal needle remains stationary relative to the main body during the transfer of the device to its retractable state, thereby preventing movement of the thread to the proximal direction, and effecting the full exposure of the thread to the patient's tissue within the intradermal channel.

Clause 8. A device according to Clause 1, wherein the displacement mechanism is configured to apply a force on a proximal end of the thread, thereby to cause a displacement of the thread such that a length of about 3 mm to about 20 mm of the thread at its distal end protrudes from the distal end of the cannula.

Clause 9. A device according to Clause 8, wherein a length of about 5 mm to about 10 mm of the thread at its distal end protrudes from the distal end of the cannula.

Clause 10. The device of Clause 8, wherein the displacement mechanism comprises a push button for displacing a needle, wherein the distal end of the needle applies a pushing force on the proximal end of the thread.

Clause 11. The device of Clause 8, wherein the displacement mechanism comprises a push button for driving a piston, the piston driving a compressed gas within a chamber, and the gas applies a force on the proximal end of the thread.

Clause 12. The device of Clause 8, wherein the displacement mechanism comprises one or more gears.

Clause 13. The device of Clause 1, further comprising mechanism for fixing the device in one or more of a loading state and an insertion state.

Clause 14. The device of Clause 1, wherein one or more of: the entire device, the penetration element or the thread, come in a sterile pack.

Clause 15. A method for creating an intradermal channel in a skin tissue of a patient, with a thread therein, the method comprising: providing a device having a main body, the main body having a lumen; providing a slidable mechanism which is permanently contained within the lumen; providing a penetration element at the distal end of the device, the penetration element comprising a cannula; providing the thread within the cannula such that the entire length of the thread is occupied within the cannula; creating the intradermal channel using the penetration element; effecting a relative displacement between the thread and the cannula by the slidable mechanism, thereby to expose at least a portion of the thread to the patient's skin tissue within the intradermal channel; and withdrawing the penetration element from the intradermal channel, leaving the thread therein.

Clause 16. A method according to Clause 15, wherein the relative displacement results from a retraction of the cannula relative to the thread.

Clause 17. A method according to Clause 15, wherein the relative displacement results from pushing a proximal end of the thread relative to the cannula, causing the distal end of the thread to protrude a length of about 3 mm to about 20 mm from the distal end of the cannula to within the patient's tissue.

Clause 18. A method according to Clause 15, wherein the relative displacement results from pushing a proximal end of the thread relative to the cannula, causing the distal end of the thread to protrude a length of about 5 mm to about 10 mm from the distal end of the cannula to within the patient's tissue.

The foregoing description is provided to enable a person skilled in the art to practice the various configurations described herein. While the subject technology has been particularly described with reference to the various figures and configurations, it should be understood that these are for illustration purposes only and should not be taken as limiting the scope of the subject technology.

It is understood that the specific order or hierarchy of steps in the processes disclosed is an illustration of exemplary approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refer to only A, only B, or only C; any combination of A, B, and C; and/or at least one of each of A, B, and C.

Furthermore, to the extent that the term “include,” “have,” or the like is used in the description or the claims, such term is intended to be inclusive in a manner similar to the term “comprise” as “comprise” is interpreted when employed as a transitional word in a claim.

As used herein, the term “about” is relative to the actual value stated, as will be appreciated by those of skill in the art, and allows for approximations, inaccuracies and limits of measurement under the relevant circumstances. In one or more aspects, the terms “about,” “substantially,” and “approximately” may provide an industry-accepted tolerance for their corresponding terms and/or relativity between items, such as a tolerance of from less than one percent to ten percent of the actual value stated, and other suitable tolerances.

As used herein, the term “comprising” indicates the presence of the specified integer(s), but allows for the possibility of other integers, unspecified. This term does not imply any particular proportion of the specified integers. Variations of the word “comprising,” such as “comprise” and “comprises,” have correspondingly similar meanings.

The word “exemplary” is used herein to mean “serving as an example, instance, or illustration.” Any embodiment described herein as “exemplary” is not necessarily to be construed as preferred or advantageous over other embodiments.

A reference to an element in the singular is not intended to mean “one and only one” unless specifically stated, but rather “one or more.” Pronouns in the masculine (e.g., his) include the feminine and neuter gender (e.g., her and its) and vice versa. The term “some” refers to one or more. Underlined and/or italicized headings and subheadings are used for convenience only, do not limit the subject technology, and are not referred to in connection with the interpretation of the description of the subject technology. All structural and functional equivalents to the elements of the various configurations described throughout this disclosure that are known or later come to be known to those of ordinary skill in the art are expressly incorporated herein by reference and intended to be encompassed by the subject technology. Moreover, nothing disclosed herein is intended to be dedicated to the public regardless of whether such disclosure is explicitly recited in the above description.

Although the detailed description contains many specifics, these should not be construed as limiting the scope of the subject technology but merely as illustrating different examples and aspects of the subject technology. It should be appreciated that the scope of the subject technology includes other embodiments not discussed in detail above. In addition, it is not necessary for a method to address every problem that is solvable (or possess every advantage that is achievable) by different embodiments of the disclosure in order to be encompassed within the scope of the disclosure. The use herein of “can” and derivatives thereof shall be understood in the sense of “possibly” or “optionally” as opposed to an affirmative capability.

What is claimed is:

1. A device for creating an intradermal channel in a skin tissue of a patient with a thread therein, the device comprising:
a main body having a lumen;
a penetration element at a distal end of the main body, the penetration element comprising a cannula which is configured to contain the thread in its entirety; and
a displacement mechanism, at least a portion thereof permanently contained within the main body lumen, the displacement mechanism is configured to effect a relative motion between the cannula and the thread, thereby to expose at least a portion of the thread to a patient's tissue within an intradermal channel.

2. A device according to claim 1, wherein the penetration element is separable from the main body of the device.

3. A device according to claim 1, wherein the penetration element is separable from a slidable portion within the main body of the device.

4. A device according to claim 3, wherein the penetration element is attachable by means of a luer-lock pair to a distal end of the slidable portion which is contained within the main body.

5. A device according to claim 4, wherein the slidable portion is movable along a longitudinal needle between an extended state and a retracted state of the device.

6. A device according to claim 5, wherein the connection of the penetration element to the slidable portion is performed when the device is in its extended state, and wherein a transfer of the device to its retractable state fully exposes the thread to the patient's tissue within the intradermal channel.

7. A device according to claim 6, wherein the longitudinal needle remains stationary relative to the main body during the transfer of the device to its retractable state, thereby preventing movement of the thread to the proximal direction, and effecting full exposure of the thread to the patient's tissue within the intradermal channel.

8. A device according to claim 1, wherein the displacement mechanism is configured to apply a force on a proximal end of the thread, thereby to cause a displacement of the thread such that a length of about 3 mm to about 20 mm of the thread at its distal end protrudes from the distal end of the cannula.

9. A device according to claim 8, wherein a length of about 5 mm to about 10 mm of the thread at its distal end protrudes from the distal end of the cannula.

10. The device of claim 8, wherein the displacement mechanism comprises a push button for displacing a needle, wherein the distal end of the needle applies a pushing force on the proximal end of the thread.

11. The device of claim 8, wherein the displacement mechanism comprises a push button for driving a piston, the piston driving a compressed gas within a chamber, and the gas applies a force on the proximal end of the thread.

12. The device of claim 8, wherein the displacement mechanism comprises one or more gears.

13. The device of claim 1, further comprising mechanism for fixing the device in one or more of a loading state and an insertion state.

14. The device of claim 1, wherein one or more of: the entire device, the penetration element or the thread, come in a sterile pack.

15. A method for creating an intradermal channel in a skin tissue of a patient, with a thread therein, the method comprising:
providing a device having a main body, the main body having a lumen;
providing a slidable mechanism which is permanently contained within the lumen;
providing a penetration element at the distal end of the device, the penetration element comprising a cannula;
providing the thread within the cannula such that the entire length of the thread is occupied within the cannula;
creating the intradermal channel using the penetration element;
effecting a relative displacement between the thread and the cannula by the slidable mechanism, thereby to expose at least a portion of the thread to the patient's skin tissue within the intradermal channel; and
withdrawing the penetration element from the intradermal channel, leaving the thread therein.

16. A method according to claim 15, wherein the relative displacement results from a retraction of the cannula relative to the thread.

17. A method according to claim 15, wherein the relative displacement results from pushing a proximal end of the thread relative to the cannula, causing the distal end of the thread to protrude a length of about 3 mm to about 20 mm from the distal end of the cannula to within the patient's tissue.

18. A method according to claim 15, wherein the relative displacement results from pushing a proximal end of the thread relative to the cannula, causing the distal end of the thread to protrude a length of about 5 mm to about 10 mm from the distal end of the cannula to within the patient's tissue.
